(19) United States
(12) Patent Application Publication
(10) Pub. No.: US 2008/0299270 A1
(43) Pub. Date: Dec. 4, 2008

(54) Fat Taste Receptors and Their Methods of Use

(75) Inventors: Sami Damak, Epalinges (CH); Johannes Le-Coutre, Pully (CH); Carole Bezencon, Riex (CH); Cristina Cartoni, Lausanne (CH)

Correspondence Address: BELL, BOYD & LLOYD LLP, P.O. Box 1135, CHICAGO, IL, 60690, US

(73) Assignee: NESTEC S.A., Vevey (CH)

(21) Appl. No.: 11/996,935

(22) PCT Filed: Jul. 10, 2006
(86) PCT No.: PCT/EP2006/064043

§ 371 Date: Jul. 28, 2008

Related U.S. Application Data
(60) Provisional application No. 60/704,021, filed on Jul. 28, 2005.

(51) Int. Cl. G01N 33/02; C07K 14/705; C12N 5/06; G01N 33/68; C12Q 1/00

(52) U.S. Cl. 426/231; 530/350; 435/325; 436/86; 435/4

(57) ABSTRACT

Fat taste receptors and methods for using the receptors to screen compounds that mimic fat taste are disclosed. The receptors can include G-protein coupled receptor proteins, such as GPR40 or GPR120. New methods for preparing foods are also disclosed that involve testing compounds in the assay system and then incorporating into foods the identified nonfat compounds that have the taste of fat.

### BACKGROUND OF THE INVENTION

The detection of fat in the mouth has traditionally been considered to rely on texture, viscosity and smell. However, some fat replacers, which mimic these qualities do not entirely mimic the mouth sensation and pleasure of fat.

Partly for this reason, it is thought that there may be a fat taste. fMRI studies have shown that vegetable oil stimulates the taste areas of the human cortex and nerve recordings in rats have shown that free fatty acid (FFA) application to the tongue stimulates the lingual branch of the glosso-pharyngeal nerve. This suggests that the fat sensation has an extra-trigeminal component. It has also been observed that isolated rat taste cells respond to medium and long chain FFAs by inhibiting a delayed rectifying potassium channel. Thus, several lines of evidence suggest that medium and long chain FFA are capable of eliciting fat taste.

Systems for screening compounds that elicit a fat taste but which are not themselves fat are needed in the food industry. Such systems could be used to identify compounds that can replace fat in foods thereby providing healthier foods having fewer calories but that retain desirable flavor characteristics.

### SUMMARY OF THE INVENTION

A functional fat taste receptor comprising a protein expressed from recombinant DNA molecule is disclosed. The protein can be a portion of a G-protein coupled receptor protein, such as GPR120, GPR40 or both. The fat taste receptor can be expressed in a cell from a recombinant DNA molecule. Cells containing fat taste receptor protein genes are also contemplated.

In an embodiment, an assay system is provided that incorporates functional fat taste receptors. The assay system can be used to identify compounds that have the taste of fat, including both fat and non-fat compounds.

In an embodiment, the assay system generates a measurable response to compounds having the taste of fat, such as upon the addition of a fat, including free fatty acids, such as long chain and medium chain fatty acids.

A method for identifying a compound that tastes like fat is also disclosed. The method can be implemented by cloning a protein component of a fat taste receptor, expressing the cloned protein and forming a fat taste receptor. The fat taste receptor can be added to a system capable of generating a response when a test compound binds to the receptor. When a response is detected, the compound is identified as having the taste of a fat.

A method for preparing a food product is also disclosed. The method can be implemented by preparing a fat taste receptor assay system and using the system to identify a nonfat compound that tastes like fat and incorporating the identified nonfat compound into a food.

Additional features and advantages of the present invention are described in, and will be apparent from, the following Detailed Description of the Invention and the figures.

### BRIEF DESCRIPTION OF THE FIGURES

FIG. 1A shows a pattern of cells from a section of the circumvallate taste papilla of a transgenic mouse. The cells in the section express green fluorescent protein (GFP) under the control of a Trpm5 promoter.

FIG. 1B shows an immunochemical staining pattern of the same section of the circumvallate papilla as in FIG. 1A, stained with a GPR120-specific antibody and detection with Cy3.

FIG. 1C illustrates an overlap of FIG. 1A and FIG. 1B in which there is about a 95% co-localization of GPR120 and Trpm5 in the taste cells.

FIG. 2 provides a schematic illustration of the distribution of sweet-responsive, bitter-responsive and umami-responsive taste receptor cells in the taste bud.

FIG. 3A shows a pattern of cells from a section of the circumvallate papilla of a transgenic mouse. The cells in the section express green fluorescent protein (GFP) under the control of a Trpm5 promoter.

FIG. 3B shows an immunochemical staining pattern of the same section of the circumvallate papilla as in FIG. 3A, stained with a GPR40-specific antibody and detection with Cy3.

FIG. 3C illustrates an overlap of FIG. 3A and FIG. 3B in which there is partial co-localization of GPR40 and Trpm5.

FIG. 4 shows preference ratios and intake volumes of fat emulsions by mice. A preference ratio of 0.5 indicates indifference, above 0.5 indicates preference and below 0.5 indicates avoidance. Both preference and intake of fat are diminished in the GPR40 KO mice compared to control mice. The stars show the concentrations of lipids at which the differences of intake or preference between knockout and control mice are statistically significant.

### DETAILED DESCRIPTION OF THE INVENTION

The present application is based, in part, on the discovery of fat taste receptors and the proteins that make up fat taste receptors. The proteins are members of a class of proteins known as G-protein coupled receptors or (GPCRs) and include GPR40 and GPR120. The inventors have shown that these receptors are present in taste tissue in cells that express Trpm5, a key taste signal transduction protein. Furthermore they have shown that mice lacking a functional GPR40 gene (GPR40 knockout mice) have diminished preference for and intake of corn oil, Intralipid, lauric acid and oleic acid. Together, these data demonstrate that GPR120 and GPR40 are fat taste receptors and can be utilized as a screening tool for compounds that mimic fat taste. Such compounds can be incorporated into foods as fat replacers.

Fat taste receptor proteins have been identified, in part, by analogy to the bitter, sweet and umami compound taste phenomenon in which the taste sensation is initiated by tastants binding to G-protein coupled receptors (GPCRs). In addition, it has been observed that GPR40 and GPR120, two GPCRs, are activated in vivo and in vitro by long and medium chain fatty acids. GPR40 is mainly expressed in the pancreas and stimulates insulin secretion in response to medium and long chain free fatty acids. GPR120 is expressed in the intestines and lungs and upon stimulation by long chain fatty acids leads to increased GLP1 secretion. The two receptors have partially overlapping but not identical ligand binding spectra. GPR40 appears to bind strongly to medium chain fatty acids (C₁₀₋₁₂) and also to saturated (C₁₄₋₁₆) and unsaturated (C₁₈₋₂₀). GPR120 does not appear to bind to (C₁₀₋₁₂) fatty acids strongly, and appears to bind saturated and unsaturated (C₁₄₋₂₀).

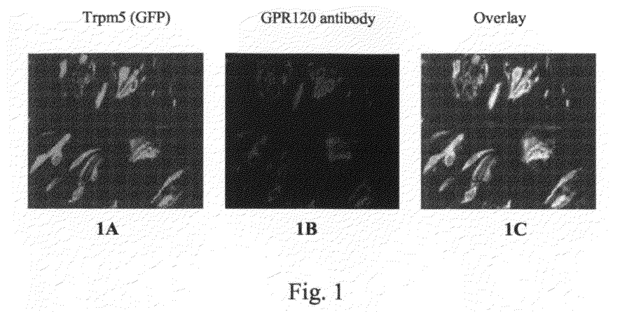

GPR40 and GPR120 are identified as being involved in fat taste receptors based, in part, on their expression in taste tissue. Using immunochemical staining techniques, the inventors have found that GPR120 is strongly expressed in many taste receptor cells in the mouse circumvallate, foliate and fungiform taste papillae. FIG. 1 illustrates a typical immunostaining pattern of a section from the circumvallate papilla, with several cells per taste bud showing immunoreactivity to a GPR120-specific antibody. FIG. 1A demonstrates the overall pattern of cells which are colored because of constitutive expression of green fluorescent protein from a Trpm5 promoter. FIG. 1B demonstrates those same cells that express GPR120 on their cell surface, which are identified as based upon immunochemical staining with a GPR120-specific antibody and detection with Cy3. When the two figures are overlayed an approximately 95% co-localization of GPR120 and GFP in the cells can be observed, indicating that GPR120 is expressed in about 95% of the cells that express Trpm5.

GPR120 has also been identified in human taste cells using sequences obtained from a human taste cDNA library. The library was constructed from 200 isolated taste receptor cells from human fungiform papillae and 17,000 clones from this library were sequenced. The sequences from the library were compared with that of GPR120 and a perfect match was observed between a 152-nucleotide sequence from the library and the 3′ end of the GPR120 gene, confirming that GPR120 is expressed in human taste tissue.

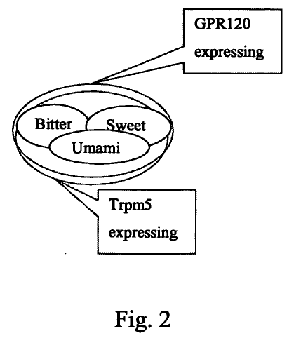

It has also been determined that GPR120 utilizes a common transduction pathway as the pathway in sweet, bitter and umami taste. Standard immunochemical staining (FIG. 1) and co-localization studies were carried out with Trpm5, a cation channel necessary for the transduction of these three taste modalities. It was observed that most if not all Trpm5-expressing cells also express GPR120. This implies that cells that respond to bitter, sweet or umami stimuli also express GPR120. A schematic representation of the distribution of sweet-responsive, bitter-responsive and umami-responsive taste receptor cells in the taste bud is illustrated in FIG. 2. As illustrated, all sweet, bitter and umami cells also express Trpm5. Because GPR120 is also expressed in almost all Trpm5 expressing cells, it is expressed in bitter, sweet and umami-responsive cells.

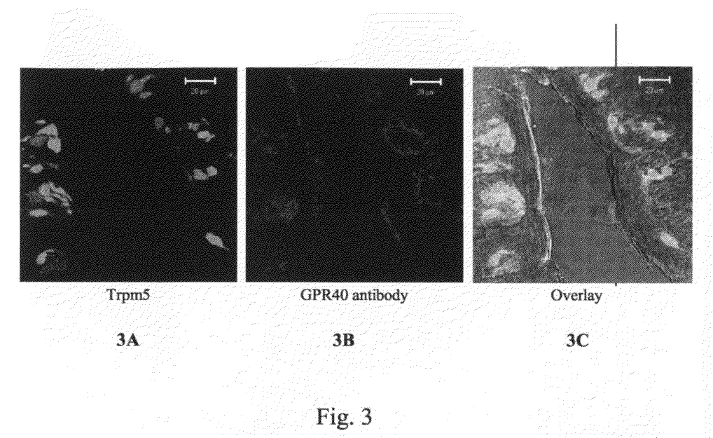

Immunohistochemical staining studies have also been used to show that GPR40 occurs in mouse taste tissue, as illustrated in FIG. 3. However, in this case only a fraction of Trpm5 expressing cells were immunoreactive to the GPR40 antibody. Co-localization studies showed that GPR40, as best seen in FIG. 3B, is expressed in a subset of Trpm5-expressing cells, and in taste cells that do not express Trpm5. FIG. 3A shows the pattern of cells in a section of circumvallate papilla from a transgenic mouse expressing GFP under the control of a Trpm5 promoter. FIG. 3B illustrates the same cell section when stained with a GPR40-specific antibody.

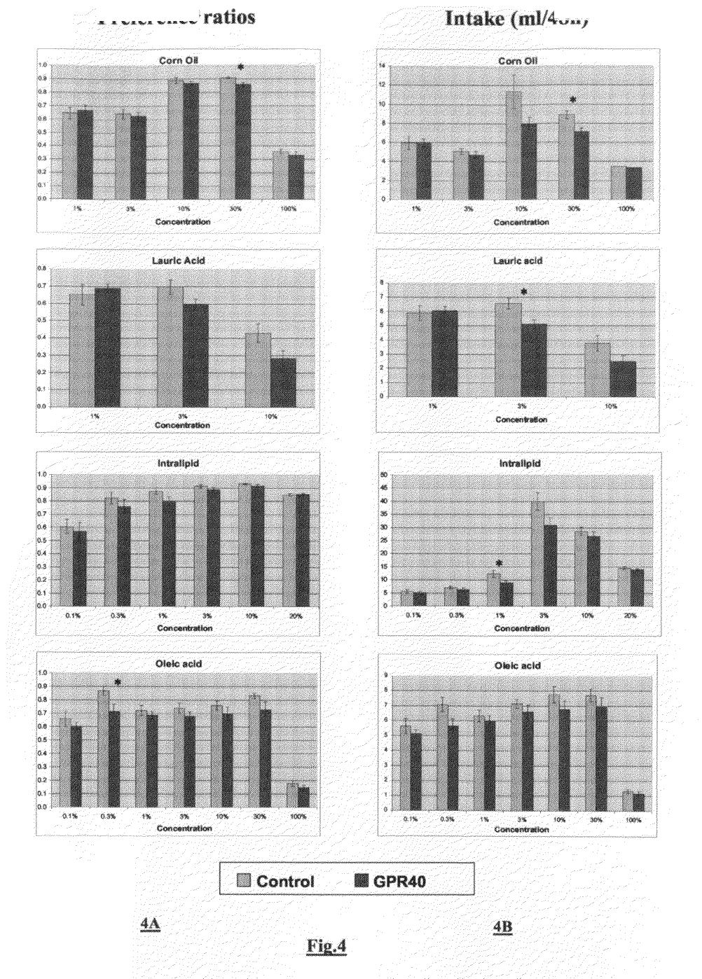

Behavioral tests in knockout mice have been used to show that GPR40 functions as a fat taste receptor. Mice lacking a functional GPR40 receptor (GPR40 knockout mice) were given the choice between two drinking bottles, one containing a fat emulsion and one containing vehicle only, as seen in FIG. 4. The fat emulsions contained corn oil, Intralipid, lauric acid or oleic acid. When compared with normal mice, the knockout animals drank less fat. The GPR40 knockout animals still had a preference for the fat emulsion compared to vehicle alone, although it was not as marked as in the normal mice. Together these studies show that GPR40 is a fat taste receptor. Because mice can still detect fat in the absence of GPR40, another receptor, probably GPR120, must also play a role in fat taste signaling.

The studies discussed above indicate that GPR120 and GPR40 are components of fat taste receptors. GPR120 is widely expressed, including in taste receptor cells expressing receptors for other taste modalities. It is likely that GPR120 is involved in the well-known modulation of other taste modalities caused by fatty acids. On the other hand, GPR40 is expressed in a different subset of taste cells and may be the main receptor that mediates the specific taste of fat.

The proteins that make up functional fat taste receptors, or a portion of them, can be cloned and expressed in non-taste cells and used in assays to identify compounds that have the taste of fat, including both fatty compounds and non-fat compounds. The assay can be a cell based assay in which the taste receptor is associated with a second component capable of providing a detectable signal in response to the binding of a compound to the taste receptor. In the assay, a test compound can be incubated with the cells under conditions to permit binding to the receptor. Binding can then be detected by determining whether a signal is generated in response to the added compound. Once compounds are identified they can be incorporated into food products to mimic the taste of fat without adding the calories and harmful health effects of fat.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. An isolated fat taste receptor.

2. The fat taste receptor of claim 1, comprising a protein expressed from recombinant DNA molecule.

3. The fat taste receptor of claim 1, comprising a protein expressed from recombinant DNA molecule wherein the protein comprises a G-protein coupled receptor.

4. The fat taste receptor of claim 1, comprising a protein expressed from recombinant DNA molecule wherein the protein comprises a portion of GPR120.

5. The fat taste receptor of claim 1, comprising a protein expressed from recombinant DNA molecule wherein the protein comprises a portion of GPR40.

6. The fat taste receptor of claim 1, comprising a protein expressed from recombinant DNA molecule wherein the protein comprises portions of GPR40 and GPR120.

7. A cell comprising a fat taste receptor comprising a protein expressed from a recombinant DNA molecule.

8. The cell of claim 7, wherein the protein is a portion of GPR40.

9. The cell of claim 7, wherein the protein is a portion of GPR120.

10. An assay system for assaying fat taste comprising a functional fat taste receptor comprising a protein expressed from a recombinant DNA molecule.

11. The assay system for assaying fat taste of claim 10, wherein the protein is a portion of GPR40.

12. The assay system for assaying fat taste of claim 10, wherein the protein is a portion of GPR120.

13. The assay system of claim 10, wherein the system generates a measurable response to a nonfat composition that has the taste of fat.

14. The assay system of claim 10, wherein the system generates a measurable response upon an addition of fat.

15. The assay system of claim 10, wherein the system generates a measurable response upon an addition of a free fatty acid.

16. The assay system of claim 10, wherein the system generates a measurable response upon an addition of a long chain free fatty acid.

17. The assay system of claim 10, wherein the system generates a measurable response upon an addition of a medium chain free fatty acid.

18. A method for identifying a compound that tastes like fat comprising:
cloning a gene that expresses a protein component of a fat taste receptor,
expressing a cloned protein,
forming a fat taste receptor,
including an expressed fat taste receptor in a system capable of generating a response upon an addition of a compound that has a fat taste,
adding a test compound to the system, and
measuring a response of the system to the added test compound.

19. The method of claim 18, wherein the protein is a G-protein coupled receptor.

20. A method for preparing a food product comprising:
preparing an assay system capable of generating a response upon an addition of a test compound that has a taste of fat,
adding a test compound to the system,
identifying a compound that tastes like fat by detecting a response in the assay system upon the addition of the compound, and
incorporating an identified test compound into a food.

21. The method of claim 20, wherein the compound is not a fat.
